(19) United States
(12) Patent Application Publication
(10) Pub. No.: US 2004/0235176 A1
(43) Pub. Date: Nov. 25, 2004

(54) Reporter gene-containing plasmid which is convertible to T-Vector and the preparation method thereof

(76) Inventors: Sangmee Ahn Jo, Kyunggi-do (KR); Chulman Jo, Kyunggi-do (KR); Byung Hak Kang, Eunpyung-ku (KR)

Correspondence Address: KLARQUIST SPARKMAN, LLP, 121 SW SALMON STREET, SUITE 1600, PORTLAND, OR, 97204, US

(21) Appl. No.: 10/489,128

(22) PCT Filed: May 15, 2002
(86) PCT No.: PCT/KR02/00910

(30) Foreign Application Priority Data
Sep. 10, 2001 (KR) 2001-55508

(51) Int. Cl. C12N 15/74

(52) U.S. Cl. 435/471

(57) ABSTRACT

The present invention relates to a plasmid, which is convertible to T-vector and useful for promoter analysis and the preparation method thereof. More particularly, the present invention relates to a plasmid which is convertible to T-vector and can be stored with ease, and particularly makes promoter analysis easy and the preparation method thereof.

### FIELD OF THE INVENTION

The present invention relates to a plasmid, which is convertible to T-vector and useful for promoter analysis and the preparation method thereof. More particularly, the present invention relates to a plasmid which is convertible to T-vector and can be stored with ease, and particularly makes promoter analysis easy and the preparation method thereof.

### BACKGROUND

Genes become synthesized as proteins mainly through two procedures of transcription and translation. It is a promoter that is responsible for transcriptional control. For the activation of a promoter, gene-specific transcription factor needs to be bound to a specific site of the promoter. This causes transcription by recruiting a RNA polymerase binding. It is important to clarify the biophysical activation of a protein synthesized from a gene and the gene expression control mechanism. Studies have been reported that core promoter site has been decided by cloning a promoter site of a gene into a vector containing reporter gene such as firefly luciferase, CAT(Chloramphenicol acetyltransferase), alkaline phosphatase and β-galactosidase, and transcription factor which reacts to a certain signal has been detected, and so did cis-acting element.

More particularly, on the study of promoter analysis, in order to decide a core promoter site, the promoter being deleted equally from the 5′ end of a cloned promoter toward the starting point of the transcription was cloned into a vector containing a reporter gene. Each promoter site was amplified by synthesizing PCR primer having the restriction enzyme recognition site at the 5′ end which is the same as restriction enzyme recognition site existing at a multi-cloning site of a major reporter vector for cloning, and cut with proper restriction enzymes after purification followed by cloning into a vector for analysis. This procedure becomes simple and easy if a T-vector containing firefly luciferase gene is used.

The gene products amplified by PCR have an additional nucleotide containing adenine at both 3′ ends because of terminal transferase activity of Taq DNA polymerase (Clark, J. M., Nucleic Acid Res., 1988, 16, 9677). A linear T-vector having additional nucleotide containing thymidine at both 3′ ends of a gene is designed for the gene product amplified by PCR to be cloned easily.

T-vector could be prepared mainly by two ways. The first way is by transferring thymidine into 3′ end following cutting with restriction enzyme, which makes gene cloning vector such as pUC series a linearized, blunt-ended plasmid. Then, deoxythymidine triphosphate (dTTP) was transmitted to the 3′ end of a linear gene by making use of either Taq polymerase (Marchunk, D., et al., Nucleic Acid Res., 1991, 19, 1154) or terminal deoxynucleotidyl transferase (Holton, T. A., et al., Nucleic Acid Res., 1991, 19, 1156). This preparation method, however, depends mostly on the activation efficiency of terminal transferase, which means incomplete vector without additional thymidine nucleotide might be generated if enzyme was not incubated in the best reaction condition. Thus, there could be found E. coli transformants transformed with a T-vector, which does not have amplified gene product, by self-ligation under the cloning procedure.

The other way of preparing T-vector is using restriction enzymes such as AspE I (Yoshikazu, I., et al., Gene, 1993, 130, 152), Hph I (David, A. M., et al., Bio/Technology, 1991, 9, 65), Mbo II and/or Xcm I. With those restriction enzymes, a gene can be cut leaving only one nucleotide at 3′ end of the gene. T-vector, in this manner, can be prepared by cutting a gene with the above restriction enzymes following the cloning oligonucleotides having two-tandemly arrayed recognition site into a mother vector, which is designed to have only thymidine at 3′ end when a gene is cut by restriction enzyme. This way, however, has a weakness that it cannot be used when the restriction enzyme recognition sites exist in mother vector. In regard to pUC-19, used mostly as a mother vector, the recognition sites of the above mentioned enzymes exist in 7 sites each of Hph I and Mbo II (Mead, D. A., et al. Bio/Technology, 1991, 9, 65). Only Xcm I has no recognition site, which encourages many scientists to focused their studies on developing T-vector by using Xcm I (Kovalic, D., et al., Nucleic Acid Res., 1991, 19, 4560; Cha, J., et al., Gene, 1993, 136, 369; Testoris, A., et al., Gene, 1994, 143, 151; Harrison, J., et al., Analytical Biochem., 1994, 216, 235; Borovkov, A. Y., et al., Biotechniques, 1997, 22, 812).

However, when a gene is cut with restriction enzyme, the separated oligonucleotide fragments therefrom are too small to confirm by checking on agarose gel electrophoresis whether the two recognition sites of the gene are completely cut or one of them left uncut. The moving distance between T-vector in which a gene is completely cut off and a vector in which one of the two recognition sites is remained uncut or a gene is uncut from the beginning is not apart resulting that unclear cut vectors are contaminated when pure T-vector is to be separated. Therefore, some vectors remained uncut by restriction enzyme could be used for cloning an amplified gene product, so that high percentage of circular T-vector having uncut oligonucleotide can be found in a transformed E. coli, which is a weakness of the second way of preparing T-vector.

Thus, the efforts have been made to develop techniques to prepare T-vectors with low rate of producing circular vectors, and with high efficiency of distinguishing DNA fragments cut off on agarose gel electrophoresis (Jo, C. and Jo, S. A., Plasmid, 2001, 45, 37).

To overcome the foregoing and other disadvantages in preparing T-vectors using restriction enzymes, we, the inventors of the present invention, have confirmed the facts that a plasmid can be clearly converted to a T-vector and T-vector for promoter analysis can be made easily when the gene which has more than 100 bp distance between two Xcm I restriction enzyme recognition sites and has adenine at the 3′ end of a gene fragment cut with Xcm I was cloned right in front of a firefly luciferase gene, by which the present invention has been accomplished.

### SUMMARY OF THE INVENTION

It is an object of this invention to provide a T-vector convertible plasmid, which is useful for promoter analysis.

It is a further object of this invention to provide an E. coli transformant, which is transformed with the above plasmid.

It is an additional object of this invention to provide a using method of the above T-vector convertible plasmid in promoter analysis.

### BRIEF DESCRIPTION OF THE DRAWINGS

FIG. 1 is a photograph of agarose gel electrophoresis showing the result of electrophoresis of glyceraldehyde 3-phosphate dehydrogenase cDNA after being amplified by PCR,

Lane 1: 100 bp DNA ladder,

Lane 2: PCR product amplified using primary cDNA isolated from rat brain tissue as a template.

FIG. 2 is a photograph of agarose gel electrophoresis showing the result of cutting T-vector cloned with the amplified gene with Sma I and HindIII,

Lane 1: λ DNA/HindIII and EcoR I molecular weight marker,

Lane 2: separated gene which was cut with Sma I and HindIII.

FIG. 3 is a photograph of agarose gel electrophoresis showing T-vector convertible plasmid pGL2-B of the present invention separated from E. coli XL1-Blue,

Lane 1: λ DNA/HindIII and EcoR I molecular weight marker,

Lane 2: T-vector convertible plasmid pGL2-B.

FIG. 4 is a diagram representing genetic map of T-vector convertible plasmid pGL2-B of the present invention constructed by using glyceraldehydes 3-phosphate dehydrogenase gene,

FIG. 5 is a photograph of agarose gel electrophoresis showing the result of T-vector convertible plasmid pGL2-X of the present invention, which was cut with Xcm I in order to be converted to T-vector,

Lane 1: λ DNA/HindIII and EcoR I molecular weight marker,

Lane 2: T-vector convertible plasmid pGL2-X,

Lane 3: pGL2-T made by cutting pGL2-X with Xcm I.

FIG. 6 is a diagram showing base sequences of the cloning part of the T-vector convertible plasmid pGL2-T of the present invention,

FIG. 7 is a photograph showing the result of agarose gel electrophoresis of AchRδ subunit promoter gene amplified by PCR,

Lane 1: 100 bp DNA ladder,

Lane 2: amplified gene product.

FIG. 8 is a photograph showing the result of agarose gel electrophoresis of T-vector which was converted from the T-vector convertible plasmid pGL2-X, and cloned with AchRδ subunit promoter gene, followed by cutting with Sty I and Pst I,

Lane 1: λ DNA/HindIII and EcoR I molecular weight marker,

Lane 2: 3,415 and 2,179 bp gene fragments produced from self-ligated plasmid,

Lane 3: 3,415, 2,312 and 393 bp gene fragments produced from plasmid in which AchRδ subunit promoter was cloned properly,

Lane 4: 100 bp DNA ladder.

FIG. 9 is a graph showing of promoter activation by neuregulin after cloning AchRδ subunit promoter gene into T-vector convertible plasmid pGL2-X.

### DETAILED DESCRIPTION OF PREFERRED EMBODIMENTS

To accomplish those objects, the present invention provides a T-vector convertible plasmid, which is useful for promoter analysis.

The present invention also provides an E. coli transformant, which is transformed with the above plasmid.

The present invention further provides a using method of the above T-vector convertible plasmid in promoter analysis.

Hereinafter, the present invention is described in detail.

In one aspect, the present invention provides a T-vector convertible plasmid, which is useful for promoter analysis.

The T-vector convertible plasmid of the present invention contains restriction enzyme recognition sites remain with only one nucleotide at 3′ end of a gene as being cut. Asp I, Hph I, Mbo II and Xcm I are the restriction enzymes mentioned above, and their recognition sites don't exist in mother vector, which makes selective use of them possible.

The T-vector convertible plasmid of the present invention contains over 100 bp base sequence between the two restriction enzyme recognition sites, and the sequence wherein adenine is located at 3′ end of a DNA fragment cut off by restriction enzyme resides right in front of a reporter gene. The above base sequence could be either a part or as a whole of any gene, or could be chosen at random from any gene. The gene could be selected from the group consisting of glyceraldehide 3-phosphate dehydrogenase, S-adenosylmethionine decarboxylase or α 2-macroglobulin receptor, etc. For a receptor gene, firefly luciferase, CAT(chloramphenicol acetyltransferase), alkaline phosphatase or β-galactosidase are preferable, and especially, firefly luciferase gene is the most preferred. In case that restriction enzyme recognition sites are exist in those reporter genes, those recognition sites need to be mutated by site-directed mutagenesis.

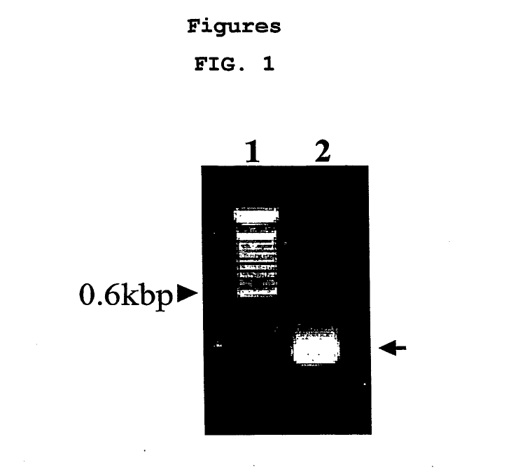

The present inventors have selected glyceraldehydes 3-phosphate dehydrogenase as a target gene containing two Xcm I restriction enzyme recognition sites, and amplified the gene by PCR. PCR was performed by using primers containing EcoR I and Xcm I recognition sites. Then, the amplified product was confirmed on agarose gel electrophoresis. As a result, 135 bp amplified product was obtained (see FIG. 1), and that PCR product was purified using PCR product purification kit, followed by cloning into pBluescript SK vector.

The above recombinant vector was cut with restriction enzymes in order to get only amplified part, leading to a preparation of T-vector convertible plasmid useful for promoter analysis by cloning the amplified product into the multi-cloning site of a vector for promoter analysis. The above recombinant vector could be used as a T-vector convertible plasmid, but other various T-vectors could also be prepared by subcloning an amplified gene into a shuttle vector or plasmids suitable for promoter analysis. The object of the present invention is to develop a T-vector suitable for promoter analysis, so that an amplified gene was subcloned into a vector for promoter analysis. Selecting a restriction enzyme which does not cut an amplified gene and a vector for promoter analysis was followed by cutting with it in a routine, and finally only amplified part of the gene was separated through agarose gel electrophoresis. Then, T-vector convertible plasmid was obtained by cloning the above amplified gene, for which the amplified gene was inserted into the corresponding restriction enzyme recognition site locating in multi-cloning site of the plasmid for promoter analysis. However, when the above plasmid converts to T-vector, Xcm I recognition site still exists in a firefly gene, which means that the reporter gene was cut off, so that it cannot be functioning as a vector anymore. Thus, the present inventors have completed the preparation of T-vector convertible plasmid for promoter analysis by making Xcm I recognition site silent mutated using site-directed mutagenesis and named it as “pGL2-X”.

The T-vector convertible plasmid useful for promoter analysis of the present invention is easily converted to T-vector with the following steps of: separating plasmid from E. coli transformant transformed with T-vector convertible plasmid, and cutting it with Xcm I restriction enzyme, and finally, separating the plasmid without inserted gene by purification steps. The plasmid of the present invention has excellency in its storage and easiness in its conversion to a T-vector. Besides, the plasmid of the present invention could be very useful for the promoter analysis by simply cloning a promoter region amplified by PCR.

This invention also provides an E. coli transformant containing T-vector convertible plasmid for promoter analysis.

The present inventors have constructed a transformant by transforming XL1-Blue using the above pGL2-X T-vector convertible plasmid, and named the E. coli transformant as “Escherchia coli (TV-3)”.

The above transformant was deposited at Korea Culture Center of Microorganisms on Jul. 23, 2001 (Accession No: KCCM 10303).

In another aspect of this invention, also provided is a using method of the above T-vector convertible plasmid in promoter analysis.

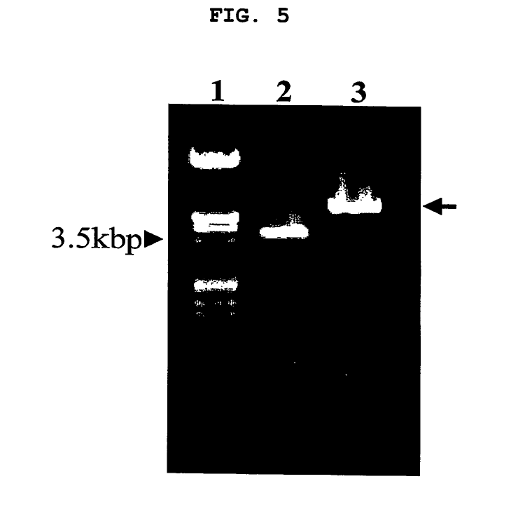

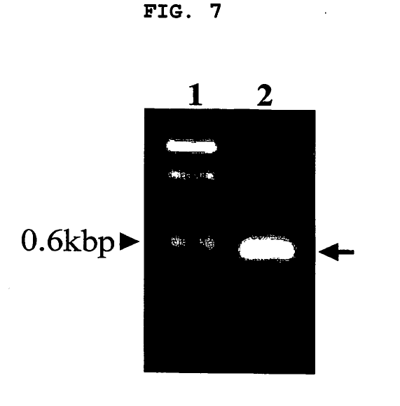

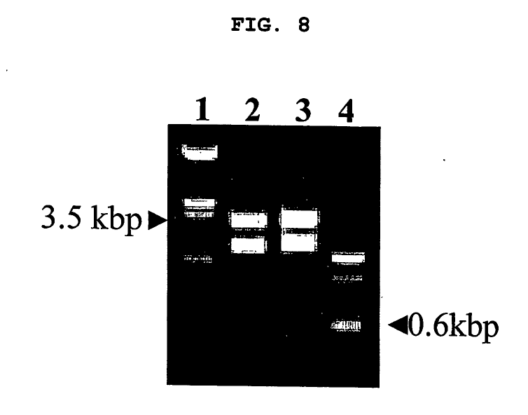

First of all, the present inventors converted the T-vector convertible plasmid of the present invention to the T-vector, and analyzed the cloning efficiency of the T-vector. The separated, purified T-vector convertible plasmid of the present invention was treated with Xcm I restriction enzyme, and performed electrophoresis. As a result, it was confirmed that the moving distance on agarose gel was different (see FIG. 5). The separated T-vector on agarose gel was purified in order to use in examining of cloning efficiency. To examine the cloning efficiency of the T-vector converted from pGL2-X plasmid with the treatment of Xcm I, acetylcholine receptor delta subunit (AchRδ) promoter was amplified by PCR, and connected to T-vector, after which cloning was done. For the amplification of the AchRδ subunit promoter, PCR was performed after synthesizing primers specific for the base sequence of the gene(SEQ. ID NO. 5 and SEQ. ID NO. 6). The amplified gene product was analyzed by electrophoresis. As a result, it was confirmed that 525 bp long AchRδ subunit promoter was amplified(see FIG. 7). The amplified product of AchRδ subunit promoter was purified and ligated into the T-vector of the present invention, and the ligate was transformed into E. coli. The transformed E. coli was cultured on LB broth, and the plasmid was separated therefrom. The separated plasmid was cut with Sty I (its recognition site exists in T-vector) and Pst I (its recognition site exists in DNA sequence of AchRδ subunit promoter) restriction enzymes to identify whether it was correctly cloned(see FIG. 8). On counting the numbers of the colonies in which AchRδ subunit promoter was not introduced and the colonies in which AchRδ subunit promoter was correctly cloned, 5 of 8 colonies have rightly cloned AchRδ subunit promoter, which presents 63% of cloning efficiency.

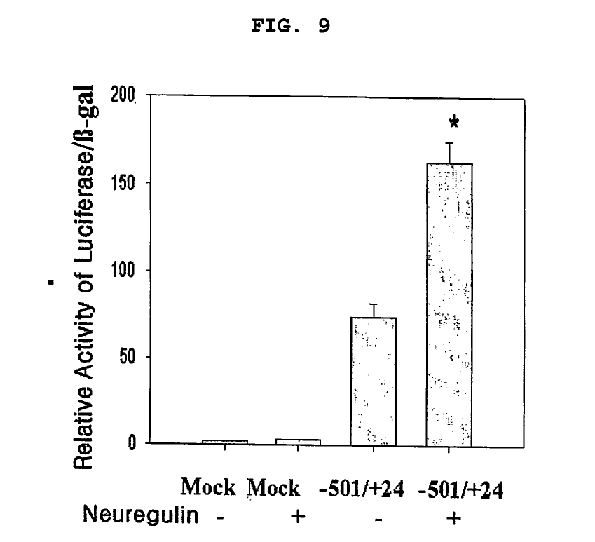

The present inventors have tested whether firefly luciferase gene constructed by the site-directed mutagenesis could be activated by the promoter cloned into the T-vector of the present invention and also could be activated by neuregulin, a kind of neuro-nutrition factor which was reported to activate AchRδ subunit promoter, in order to evaluate the activity of firefly luciferase gene existing in T-vector convertible plasmid for promoter analysis. As a result, it was confirmed that neuregulin induced approximately 2.2-fold increase in luciferase activity, which is similar to the previous report concerning AchRδ subunit promoter analysis(Si, J., et al., Journal of Biological Chemistry, 1997, 272, 10367). Thus, it was certain that mutation of luciferase gene existing in pGL2-X did not affect its activity (see FIG. 9).

### EXAMPLES

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### Gene Amplification by PCR

<1-1> Primary cDNA Synthesis of RNA Obtained from Mouse Brain

To prepare gene which might be used as a template for PCR amplification of glyceraldehydes 3-phosphate dehydrogenase gene containing two Xcm I restriction sites, the present inventors isolated total RNA from brain tissue of rat by using TRI reagent(Molecular Research Center, Inc., U.S.A.). And the primary cDNA was synthesized by adding 10 U of reverse transcriptase(Promega, U.S.A.) made from AMV(avian myeloblastosis virus) into pre-made reagent(2 μg of RNA, 1 μg of oligo(dT)₁₅, 1 mM of deoxynucleotide triphosphate). Particularly, reaction mixture except reverse transcriptase was reacted at 65° C. for 5 minutes to eliminate 2^{nd} or 3^{rd} structure of RNA. Then, this reaction mixture was dipped in ice, and reverse transcriptase was added. Finally, the above mixture was heated at 95° C. for 5 minutes to inactivate reverse transcriptase which can inhibit PCR reaction. This synthesized cDNA was used as a template for PCR reaction.

<1-2> Gene Amplification

The present inventors amplified the cDNA synthesized in <1-1> by PCR. PCR was performed in PCR buffer using 50 ng of template cDNA, 2.5 U of Tag DNA polymerase and 20 pmol of primers (SEQ. ID NO. 1 and SEQ. ID No. 2) in total volume of 50 μl (50 mM KCl, 0.1% Triton X-100, 1.5 mM MgCl₂, 150 μM dATP, dTTP, dGTP, dCTP). These primers contain EcoR I recognition site at the 5′ end in addition to Xcm I recognition site complementary to GADPH gene. Amplification was performed using a GeneAmp 2400 thermocycler(Perkin Elmer, Norwalk, Conn.) by 30 cycles as follows: a denaturing step at 95° C. for 30 seconds, a primer annealing step at 46° C. for 30 seconds and an extension step at 72° C. for 45 seconds. After PCR, the PCR product was analyzed by agarose gel electrophoresis.

As a result, it was confirmed that a 135 bp GADPH DNA fragment was amplified (FIG. 1). This amplified gene product was purified using PCR product purification kit (Qiagen, Germany), and cloned into pBluescript SK vector.

### Example 2

### Construction of Recombinant Vector

Amplified gene obtained in <Example 1> and pBluescript SK vector were cut with EcoR I. 5 U EcoR I/1 μg DNA was treated and reacted at 37° C. for 10 hours, and then the DNA fragment was purified from 1% agarose gel. 30 ng of amplified gene and 50 ng of pBluescript SK vector were ligated by using 3 U of T4 DNA ligase (Promega, U.S.A.). 30 mM Tris-HCl solution (pH 7.8) containing 10 mM MgCl₂, 10 mM DTT and 1 mM adenosine triphosphate was used as reaction buffer. Reaction was performed at 18° C. for 12-24 hours. And, E. coli XL1-Blue was transfected by heat-shock method. Particularly, 10 μl of gene solution ligated with pBluescrip SK vector was added into 100 μl of XL1-Blue solution treated with CaCl₂, and this mixture was placed on ice. 30 minutes after, the mixture was heated at 42° C. for 2 minutes, cooled for 1 minute on ice, added with 800 μl of LB medium. After 45 minutes shaking culture at 37° C., the mixture was inoculated on the MacConkey agar plate containing 50 μg/ml of ampicillin. This plate was cultured at 37° C. for 12-18 hours, and then the plasmid was isolated from white colonies.

This recombinant vector was cut with Sma I and Hind III to confirm that the amplified gene was inserted, and purified for subcloning into the plasmid which can be used for promoter analysis. Highly purified recombinant vector was treated with 5 U/1 μg DNA of Sma I and HindIII, reacted at 37° C. for 5 hours, and electrophoresis was performed for 15 minutes at 100 V with 1% agarose gel.

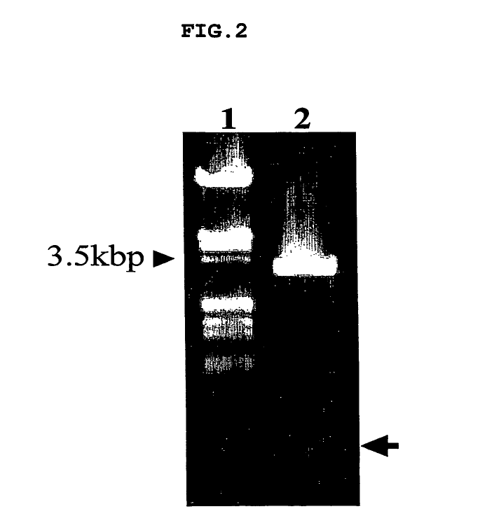

As a result, a 153 bp fragment was observed on the agarose gel (FIG. 2), indicating that amplified gene was cloned into pBluescript SK vector. This 153 bp fragment was highly purified using gel-elution kit (Qiagen, Germany).

### Example 3

### Cloning of Amplified Gene into the Plasmid for Promoter Analysis

153 bp gene containing Xcm I recognition site obtained in <Example 2> was subcloned into the plasmid for promoter analysis. As a plasmid for promoter analysis, pGL2-Basic (Promega, U.S.A.) was selected. 5 U/1 μg pGL2-Bsic of Sma I and HindIII was treated at 37° C. for 5 hours, and purified with PCR product purification kit(Qiagen, Germany), and ligated with the purified gene in <Example 2> by reaction at 16° C. for 12-18 hours in the presence of DNA ligase(3 U, Promega, U.S.A.). And then, XL1-Blue was transfected as follows. 10 μl of gene solution was added into 100 μl of XL1-Blue solution treated, and this mixture was placed on ice. 30 minutes after, the mixture was heated at 42° C. for 2 minutes, cooled for 1 minute on ice, added with 800 μl of LB medium. After 45 minutes shaking culture at 37° C., the mixture was inoculated on the LB plate containing 50 μg/ml of ampicillin. This plate was cultured at 37° C. for 12-18 hours, and then the plasmid was isolated from colonies. The plasmid was named as “pGL2-B”.

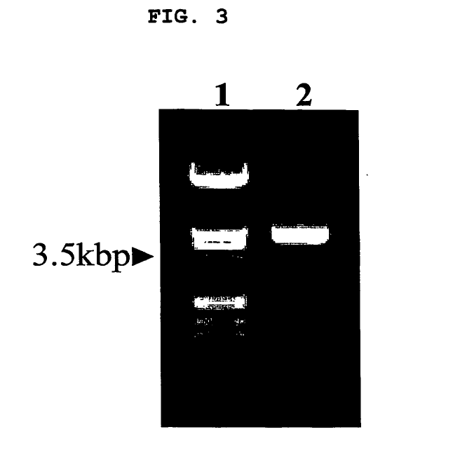

The isolated plasmid for T-vector was verified on the agarose gel (FIG. 3).

### Example 4

### Site-directed Mutagenesis of Xcm I Recognition Site within Luciferase Gene

T-vector convertible plasmid pGL2-B constructed in <Example 3> for promoter analysis is not convert to T-vector because one Xcm I recognition site is located within luciferase gene, which results in unwanted cutting of the reporter gene by Xcm I digestion. Therefore, we, the present inventors ought to perform site-directed mutagenesis to remove the Xcm I recognition site by using Transformer Site-Directed Mutagenesis Kit(Clontech, U.S.A.). Particularly, 0.1 μg of pGL2-B vector and primers(selection primer described as SEQ. ID NO. 3 and mutagenic primer described as SEQ. ID NO. 4, 0.1 μg each) were mixed, and this mixture was boiled at 100° C. for 3 minutes and cooled. Ligation was performed at 37° C. for 2 hours with 3 U of T4 DNA polymerase and 5 U of T4 DNA ligase. The ligate was purified by ethanol precipitation, restricted with Sal I (5 U) at 37° C. for 2 hours, and then transformed into E. coli BMH 71-18 mutS. Transformed E. coli was overnight shaking-cultured at 37° C., and the plasmid was obtained by using mini-prep kit(Qiagen, Germany). The plasmid was treated with Sal I again for secondary selection, and transformed into E. coli XL1-Blue. Transformed E. coli was inoculated on the plate and cultured at 37° C. for 12-18 hours. The induction of site-directed mutagenesis was confirmed by treatment of Xcm I to plasmids isolated from colonies on the plate.

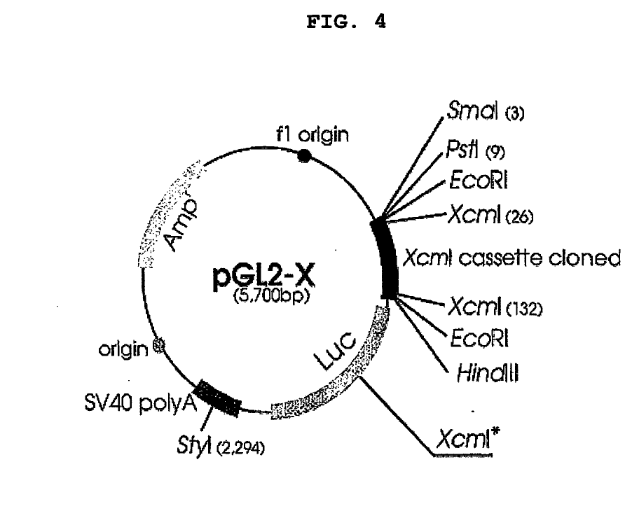

As a result, pGL2-B vector containing luciferase gene which is not cut by Xcm I was selected, and named as “pGL2-X”(FIG. 4)

The T-vector convertible plasmid, pGL2-X was transformed into E. coli XL1-Blue, and the transformant was deposited at Korea Culture Center of Microorganisms on Jul. 23, 2001 (Accession No: KCCM 10303).

### Experimental Example 1

### Conversion of T-Vector Convertible Plasmid

In order to convert T-vector convertible plasmid cloned with 153 bp gene containing Xcm I recognition sites into the T-vector, purified pGL2-X was reacted at 37° C. for 7 hours with 5 U Xcm I/1 μg DNA, and then electrophoresis was performed with 1% agarose gel.

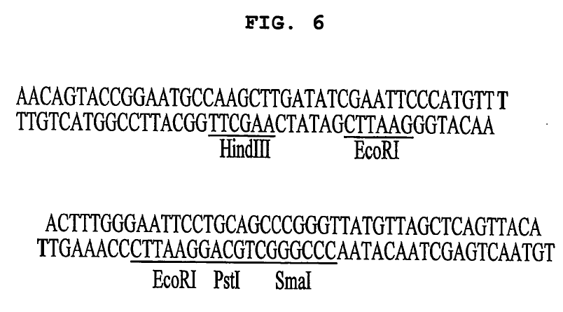

As a result, when the pGL2-X was cut off, it was confirmed that the length of movement was different on the agarose gel (FIG. 5). Nucleotide sequences of multicloning site of T-vector (pGL2-T) converted from pGL2-X were shown in FIG. 6(SEQ. ID NO. 7, 8, 9 and 10).

### Experimental Example 2

### Cloning of T-Vector Converted from T-Vector Convertible Plasmid

To examine the cloning efficiency of pGL2-T, the promoter region of mouse acetylcholine receptor delta subunit(AchRδ) gene was amplified by PCR and ligated into the pGL2-T, and then the ligate was transformed into E. coli XL1-Blue. For the amplification of AchRδ subunit promoter, PCR was performed using primers(SEQ. ID NO 5 and 6) specific for the gene. PCR was performed in PCR buffer using 50 ng of pcDNA/AchRδ as template, 20 pmole of primers and 2.5 U of Tag DNA polymerase(Promega, U.S.A.) in 50 μl of reaction mixture(10 mM of Tris-HCl(pH 9.0), 50 mM of KCl, 0.1% Triton X-100, 1.5 mM of MgCl₂, 150 μM of dNTPs). Amplification was performed using GeneAmp 2400 thermocycler(Perkin Elmer, U.S.A.) by 30 cycles as follows: a denaturing step at 95° C. for 30 seconds, a primer annealing step at 49° C. for 30 seconds and an extension step at 72° C. for 1 minute. After electrophoresis, amplified gene was purified from 1% agarose gel using PCR product purification kit (Qiagen, Germany).

As a result, it was confirm that 525 bp AchRδ subunit promoter was amplified(FIG. 7). 30 ng of purified AchRδ subunit promoter and 50 ng of T-vector constructed in <Example 1> were ligated using T4 DNA ligase (3 U, Promega, U.S.A.), and the ligate was transformed into E. coli XL1-Blue. Transformed E. coli was smeared onto the LB plate. To identify correct recombinant clones, each of 8 colonies randomly chosen was inoculated into 3 ml of LB broth containing ampicillin(50 μg/ml) and the purified plasmid was digested with Sty I and Pst I restriction enzymes.

As a result, it was confirm that five out of eight colonies contained AchRδ subunit promoter rightly cloned, indicating the cloning efficiency was 63%(FIG. 8).

### Experimental Example 3

### Analysis of Luciferase Gene Activity Using pGL2-T/AchRδ Subunit Promoter Plasmid

The present inventors analyzed whether the luciferase gene is activated by the promoter cloned into T-vector of the present invention and neuregulin which has been reported to activate AchRδ subunit promoter, and estimated the activity of luciferase gene exists in T-vector convertible plasmid for promoter analysis. Particularly, 1 μg of each pCMVβ-gal plasmid and pGL2-T/AchRδ subunit promoter plasmid or pGL2-Basic(Mock) vector were transiently transfected into C2C12 cell line cultured in DMEM/10% FBS using lipofectamine(Gibco, U.S.A.). After adding neuregulin, luciferase(Luciferase assay kit, Promega, U.S.A.) and β-galactosidase activities were assayed.

As a result, neuregulin induced approximately 2.2-fold increase in luciferase activity (FIG. 9), which is similar to the previously reported. Thus, mutation of luciferase gene in pGL2-X did not affect its activity.

### INDUSTRIAL APPLICABILITY

As shown above, the T-vector convertible plasmid of the present invention is easily convertible to T-vector and stored without difficulty. It also makes the complicated genetic recombination procedures of conventional promoter analysis simple and easy. So, the T-vector convertible plasmid of the present invention could be very useful for promoter analysis.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A promoter analysis method comprising: constructing a T-vector convertible plasmid having a reporter gene, wherein a DNA molecule is amplified by PCR, wherein at least 100 bp are present between two, restriction enzyme recognition sites in the DNA molecule, wherein cutting with the restriction enzyme that recognizes the restriction enzyme recognition sites leaves a thymidine at both 3′ ends, wherein the amplified DNA molecule is cloned into the vector, thereby generating a recombinant vector, thus, making the T-vector convertible plasmid with a reporter gene;
transforming bacteria with the recombinant vector, thereby generating transformants;
recovering the recombinant vector from the transformants;
obtaining the T-vector with a reporter gene by cutting the recombinant vector with the restriction enzyme that recognizes the restriction enzyme recognition site;
cloning a promoter region amplified by PCR into the which makes use of a T-vector convertible plasmid, wherein the T-vector convertible plasmid containing DNA in the length of over 100 bp, reporter gene located at the downstream of the above DNA and restriction enzyme recognition sites located at both ends and cut with leaving thymidine at both 3′ ends of the DNA; and
determining an amount of activity of the reporter gene, thereby analyzing an amount of promoter activity of a promoter.

2. The promoter analysis method according to claim 1, wherein the DNA molecule is obtained from a glyceraldehyde 3-phosphate dehydrogenase, S-adenosylmethionine decarboxylase or α2-macroglobulin receptor gene.

3. The promoter analysis method according to claim 1, wherein the restriction enzyme is AspE I, Hph I, Mbo II or Xcm I.

4. The promoter analysis method according to claim 3, wherein the restriction enzyme is Xcm I.

5. The promoter analysis method according to claim 1, wherein the reporter gene is firefly luciferase, CAT (chloramphenicol acetyltransferase), alkaline phosphatase or β-galactosidase.

6. (canceled)

7. The promoter analysis method according to claim 1, wherein the T-vector convertible plasmid is pGL2-X, wherein Xcm I is the restriction enzyme recognition site and firefly luciferase gene is the reporter gene.

8. An E. coli transformant transformed with T-vector convertible plasmid pGL2-X and deposited at (Korea Culture Center of Microorganisms (KCCM) Accession Number 10303).

9. (canceled)


SEQUENCE LISTING (10 sequences)

SEQ ID NO: 1 - DNA, 23 bp, Artificial Sequence
  glyceraldehyde 3-phosphate dehydrogenase primer 1
ggaattcccatgtttgtgatggg

SEQ ID NO: 2 - DNA, 23 bp, Artificial Sequence
  gyceraldehyde 3-phosphate dehydrogenase primer 2
ggaattcccaaagttgtcatgga

SEQ ID NO: 3 - DNA, 22 bp, Artificial Sequence
  selection primer
ctggatccgtcagccgatgccc

SEQ ID NO: 4 - DNA, 22 bp, Artificial Sequence
  mutagenic primer
ttgttccatttcatcacggttt

SEQ ID NO: 5 - DNA, 18 bp, Artificial Sequence
  Acethylcoline receptor delta subunit primer
actcattctgtagaccag

SEQ ID NO: 6 - DNA, 18 bp, Artificial Sequence
  Acethylcoline receptor subunit primer 2
cccttcagcctgttgctg

SEQ ID NO: 7 - DNA, 43 bp, Artificial Sequence
  pGL2-T multi-cloning site
aacagtaccggaatgccaagcttgatatcgaattcccatgttt

SEQ ID NO: 8 - DNA, 42 bp, Artificial Sequence
  pGL2-T multi-cloning site
ttgtcatggccttacggttcgaactatagcttaagggtacaa

SEQ ID NO: 9 - DNA, 44 bp, Artificial Sequence
  pGL2-T multi-cloning site
actttgggaattcctgcagcccgggttatgttagctcagttaca

SEQ ID NO: 10 - DNA, 45 bp, Artificial Sequence
  pGL2-T multi-cloning site
ttgaaacccttaaggacgtcgggcccaatacaatcgagtcaatgt